(19) United States
(12) Patent Application Publication
(10) Pub. No.: US 2008/0228203 A1
(43) Pub. Date: Sep. 18, 2008

(54) SYSTEM AND METHOD FOR TRANSLUMENAL CLOSURE IN NATURAL ORIFICE SURGERY

(75) Inventors: Stephen Graham Bell, Roma (IT); Wayne A. Node, Mission Viego, CA (US)

Correspondence Address: ROGITZ & ASSOCIATES, 750 B STREET, SUITE 3120, SAN DIEGO, CA, 92101, US

(73) Assignee: Minos Medical

(21) Appl. No.: 12/047,509

(22) Filed: Mar. 13, 2008

Related U.S. Application Data
(60) Provisional application No. 60/895,086, filed on Mar. 15, 2007.

(51) Int. Cl. A61B 17/10; A61B 17/04; A61B 17/064

(52) U.S. Cl. 606/144; 128/898; 606/219; 606/216

(57) ABSTRACT

To close a translumenal hole made in the stomach (or colon, etc.) pursuant to natural orifice surgical treatment of tissue in the peritoneal cavity, one or more guides are engaged with the hole, and the guides can be pulled to purse together tissue on opposite sides of the hole. A closure device is then advanced along the guides over the pursed tissue to adhere the tissue together using heat, staples, sutures, etc.

This application claims priority from U.S. provisional patent application 60/895,086, filed Mar. 15, 2007, incorporated herein by reference.

### I. FIELD OF THE INVENTION

The present invention relates generally to closing translumenal fenestrations in the alimentary tract such as the stomach, colon, or bladder and also to close such fenestrations in, e.g., the vagina and uterus pursuant to natural orifice surgery.

### II. BACKGROUND OF THE INVENTION

Among the applications of natural orifice surgery are procedures involving accessing the peritoneal cavity through the mouth, esophagus, and stomach to perform various tasks, e.g., gall bladder treatment, etc. Such procedures require a translumenal, i.e., fenestrating the stomach to form a hole through which a surgical instrument can be advanced into the peritoneal cavity. As recognized herein, tightly and securely closing such holes after the task is performed is even more critical than other surgical closures because the stomach contains highly acidic contents which, if leaked out of the stomach, can cause peritonitis or other complications.

### SUMMARY OF THE INVENTION

A method includes engaging one or more guides with stomach tissue adjacent a hole in the tissue. The guides extend into the esophagus and preferably out of the mouth. A closure device is advanced through the mouth and esophagus into the stomach over the guide. The guide is retracted to move tissue contiguous to the hole into a pursed configuration and then the closure device is actuated to hold the tissue in the pursed configuration.

The closure device may use non-mechanical means to fuse tissue in the pursed configuration. Or, the closure device may use mechanical fasteners such as, e.g., sutures, staples, T-anchors, etc. to hold tissue in the pursed configuration.

In another aspect, an assembly includes a closure device configured for adhering tissue together using mechanical or non-mechanical means and one or more guides configured for engagement with body tissue while extending out of a patient's natural orifice. The closure device is formed with a guide channel for receiving the guide therein to facilitate advancing the closure device through a natural orifice to a tissue opening to be closed.

In still another aspect, a method for closing a hole in tissue includes advancing at least one guide through a natural orifice to the hole, engaging the guide with tissue adjacent the hole, and advancing a closure device through a natural orifice over the guide to the tissue adjacent the hole. The method also includes moving the guide to purse tissue together in a pursed configuration and actuating the closure device to adhere tissue in the pursed configuration.

The details of the present invention, both as to its structure and operation, can best be understood in reference to the accompanying drawings, in which like reference numerals refer to like parts, and in which:

### BRIEF DESCRIPTION OF THE DRAWINGS

FIG. 1 is a perspective view showing an endoscope and closure tool advanced through the mouth into the stomach;

FIG. 2 is a perspective view of the guides, with the proximal segments of the guides cut away;

FIG. 3 is a perspective view of the closure tool being advanced over the guides;

FIG. 4 is a perspective view of the closure tool in operable position to close a translumenal;

FIGS. 5-9 are schematic views showing one non-limiting method to place the guides;

FIGS. 10-15 are schematic diagrams showing a suture placement tool and method that uses a first natural orifice to place guides and a second natural orifice through which a tissue device is guided over the guides;

FIGS. 16-19 are schematic diagrams showing a push-to-open guide placement assembly and method;

FIGS. 20-24 are schematic diagrams showing a push-to-close guide placement assembly and method; and

FIGS. 25-29 are schematic diagrams showing an alternate suture placement tool and method that uses a first natural orifice to place guides and a second natural orifice through which a tissue device is guided over the guides.

### DETAILED DESCRIPTION OF THE PREFERRED EMBODIMENT

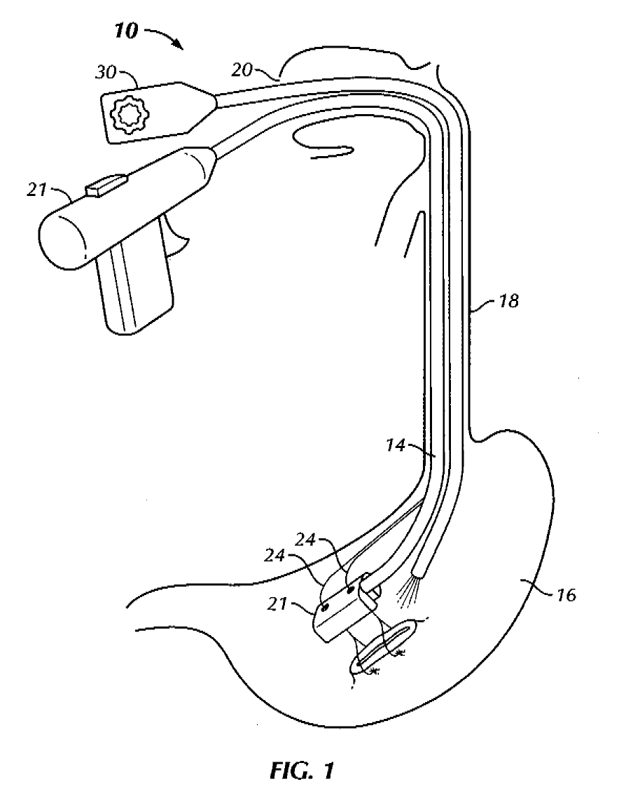

Referring initially to FIG. 1, an assembly is shown, generally designated 10, which includes a closure device 12 mounted on the distal end of a flexible hollow shaft 14 that can extend from the stomach 16 of a patient, through the esophagus 18, and out of the mouth 20 to terminate in an operating handle 21. In accordance with disclosure below, the handle 21 is operable by a surgeon to actuate the closure device 12 to adhere tissue together using mechanical or non-mechanical means. It is to be understood that present principles may apply to natural orifice closure of translumenal openings in the colon, bladder, vagina, uterus, etc.

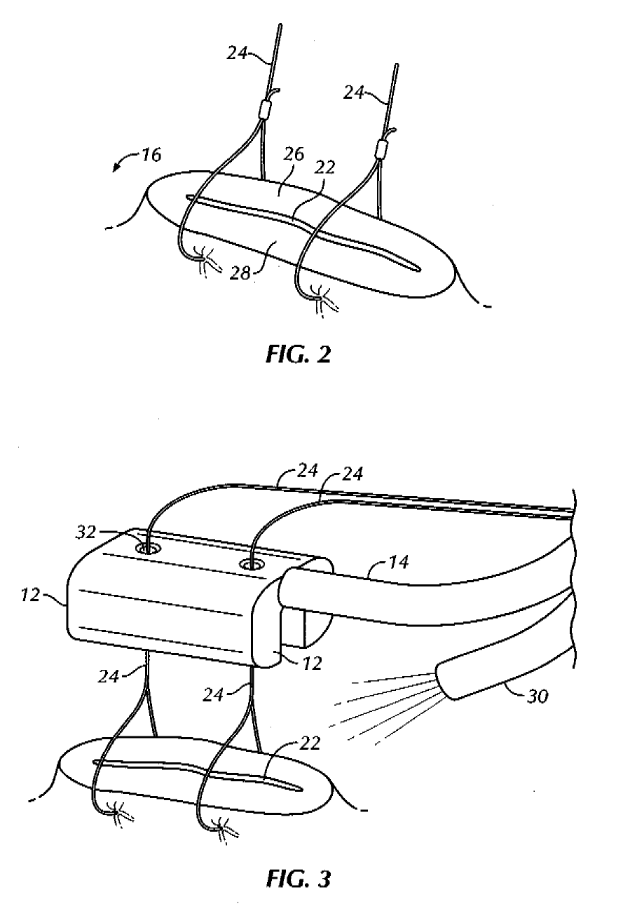

As shown in FIG. 1 and in cross-reference to FIG. 2, to guide the closure device 12 to a hole 22 to be closed in, e.g., the wall of the stomach 16, at least one and preferably two or more elongated flexible guides 24 are engaged with the tissue as set forth further below. The closure device 12 is advanced to the hole 22 over the guides 24, which may be made of suture material or other thread-like or wire-like material or which may be thin catheters as described further below. In addition to establishing means for guiding the closure device 12, the guides 24 extend away from the hole 22, in some cases out of the mouth 20, and can be tensioned to move tissue surrounding the hole 22 into the pursed configuration shown in FIG. 2, wherein tissue 26 on one side of the hole 22 is held against and facing tissue 28 on the other side of the hole 22, for purposes to be shortly disclosed.

In some embodiments, visualization of the hole 22 and closure device 12 may be provided by an endoscope 30 that may be advanced through a natural orifice such as the mouth 20. The hole 22 may be formed in the stomach 16 pursuant to a natural orifice surgical translumenal, it being understood that present principles may apply to closing other tissue holes whether formed in the stomach or whether formed deliberately or through disease or injury.

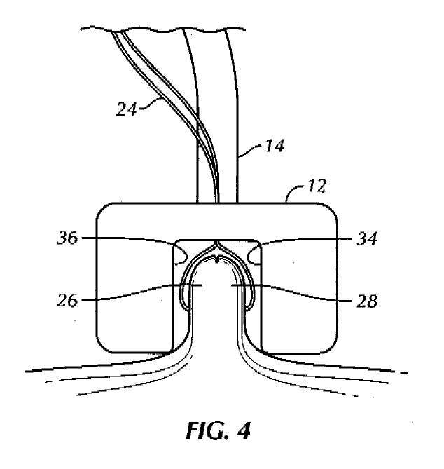

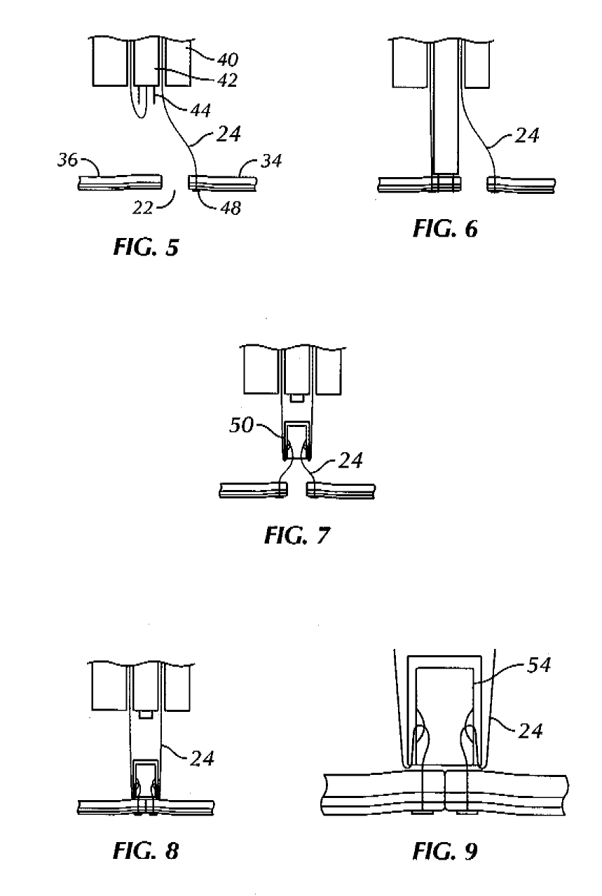

FIGS. 3 and 4 illustrate how the assembly 10 can be used to effect secure and complete closure of the hole 22. The guides 24 are advanced through the mouth 20 to the hole 22 sought to be closed, and then engaged with stomach tissue adjacent the hole. Non-limiting details of how this engagement may be effected are set forth further below. Preferably, as shown the guides 24 are passed through the tissue and form partial or complete loops as shown. The guides can pass through the entire wall of the structure, partially pass to be intramural or can be attached by grasping the tissue. Thus, the guide configuration is not limited to a complete loop but can also be a single strand attached by T-tag, or wired or other method.

The closure device 12 is formed with respective through-channels 32, and the proximal ends of the guides 24 can be positioned through respective channels 32 while the closure device 12 is outside the patient. Then, the closure device 12 can be advanced through the mouth and esophagus into the stomach over the guides 24.

As perhaps best shown in FIG. 4, the guides 24 can be retracted by a surgeon to move the tissue 26, 28 that is contiguous to the hole 22 into the pursed configuration described above. Some embodiments of the closure device 12 may include opposed closure surfaces 34, 36 as shown, between which tissue can be disposed in the pursed configuration. The guides 24 may be pulled to pull the tissue between the surfaces 34, 36 or the pursed configuration may be first established and then the guide 12 advanced over it.

With the tissue between the surfaces 34, 36, the closure device 12 is actuated to hold the tissue in the pursed configuration. In one embodiment, the closure device 12 uses non-mechanical means to hold the tissue in the pursed configuration. In non-limiting implementations the surfaces 34, 36 may pivot or otherwise move relative to each other to clamp tissue between them, and electrical leads can extend from the surfaces 34, 36 through to a source of electricity that is external to the patient to heat the surfaces 34, 36. The surfaces 34, 36 may be hollow so that they may be evacuated to further draw tissue into them. The surfaces 34, 36 are then heated to fuse clamped tissue together. Instead of rigid surfaces 34, 36, the closure device 12 may include a flexible conductive loop of, e.g., wire. By “fuse” is meant tissue welding using principles of bipolar electrocautery, ultrasonic tissue welding, laser tissue welding, etc. in addition to heat fusion, in which case the surfaces 34, 36 are configured as electrocautery arms, ultrasonic transducers, laser emitters, etc.

Or, the closure device 12 may use mechanical closure means such as sutures, staples, T-anchors, and the like. For instance, when staples are used, the closure device is a stapler and staples may be held adjacent one surface 34 from whence they may be pushed through tissue to the opposite surface 36, which establishes a staple anvil, by appropriately manipulating the handle 21 (FIG. 1) in accordance with surgical stapling principles known in the art. T-anchors likewise may be deployed through the tissue to hold it in the tightly closed pursed configuration shown.

While FIGS. 3 and 4 illustrate tissue being inverted serosa to serosa from inside the stomach, as an alternative the guides may extend out of, e.g., the anus or urethra and the closure device advanced along the guides to evert the stomach tissue mucosa to mucosa.

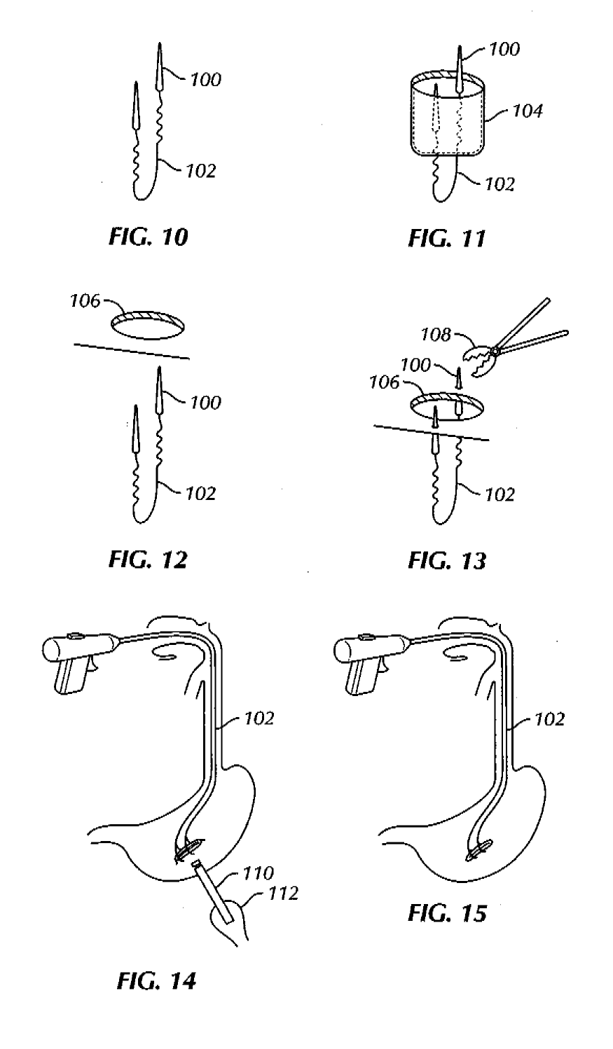

FIGS. 5-9 show one non-limiting structure and method for deploying the guides, it being understood that other structures and methods may be used. For example, the “Eagle Claw” system made by Olympus Medical Systems Corp. of Tokyo may be used. Details of suturing structures and methods provided by Olympus may be found in USPP 2007/0260214 and USPP 2007/0112362, incorporated herein by reference. Or, the g-prox system and method provided by USGI Medical may be used. Details of suturing structures and methods provided by USGI Medical may be found in USPP 2006/0271101 and USPP 2006/0271073, incorporated herein by reference.

Details of the structure and method shown in FIGS. 5-9 may be found in the following publications, incorporated herein by reference: Fritscher-Ravens et al., “Transgenic Gastropexy and Hiatal Hernia Repair for GERD under EUS Control: Porcine Model”, Gastrointestinal Endoscopy, 59:89-95 (2004); Sclabas et al., “Endoluminal Methods for Gastrotomy Closure in Natural Orifice TransEnteric Surgery (NOTES)”, Surgical Innovation, vol. 13, no. 1, pages 23-30 (March 2006).

In FIG. 5, a hollow deployment mechanism 40 with hollow inner tube 42 is advanced through the mouth into the stomach. A first stitch of the guide 14 is placed through a very small stitch hole made by a reciprocating needle 44 in the deployment mechanism 40 through the full thickness of the stomach wall. The needle 44 is then positioned to place a second stitch. A tag 48 is shown that has been inserted by means of the tip of the needle onto the distal (peritoneal) end of the stitch, to hold the stitch in place.

FIG. 6 shows that a second stitch is placed through the full thickness of the stomach wall on the side of the hole 22 that is opposite the first stitch. A thread-locking device 50 (FIG. 7) is advanced to hold the stitches of the guide together and the opening 22 closed by pulling the guide (FIG. 8). FIG. 9 shows that after being used to guide the closure device 12 to the hole to adhere the tissue in the pursed configuration as described above, if desired the guides 24 may be cut by a thread cutter device 54.

The methods above apply to full thickness passing of the guide or partial passing into one of the layers of the structure to be closed, mucosa, muscle, serosa, etc.

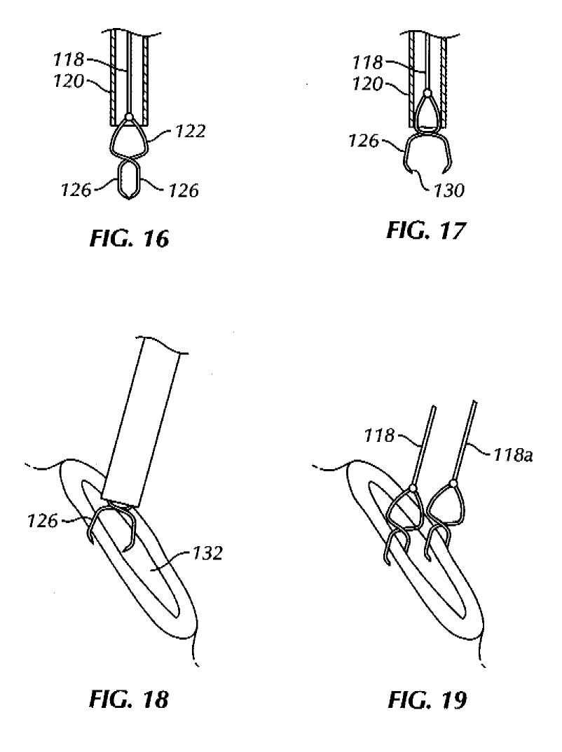

FIGS. 10-15 show an alternate guide-fixing system and method. Two axially rigid sharp needles 100 are connected at their blunt ends to a guide 102 such as a suture thread or wire. The guide 102 may be collapsed as shown. FIG. 11 shows that the guide 102 with needles 100 may be loaded into a cartridge 104 which uses an externally-actuated spring or rod or other structure to urge the needles 100 into tissue as described further below.

The cartridge 104 with guide 102 may be advanced through an endoscope to the exterior of the stomach adjacent a hole 106 (FIG. 12, omitting the cartridge 104 for clarity) to be closed. The guide assembly shown in FIGS. 10-15 may be advanced through a natural orifice other than the mouth, e.g., the urethra and then through the bladder, and out of a hole in the bladder to the exterior of the stomach, it being recognized that a bladder hole may pose less complications of repair than a stomach hole. Or, the guide assembly may be advanced through the anus and out of a hole in the intestines into the peritoneal cavity to the location shown in FIG. 12. Yet again, the guide assembly may be advanced into the peritoneal cavity percutaneously through, e.g., a laparoscopic port.

As shown in FIG. 13, the needles 100 are urged to puncture the stomach from the outside on opposite sides of the hole 106 from each other. Graspers 108 that may be advanced into the stomach through the mouth are used to grasp the needles, pulling them and the guide 102 into the stomach and preferably entirely out of the mouth as shown in FIG. 14. To guide needle puncturing, an endoscope 110 that may, e.g., be advanced through the bladder 112 into the peritoneal cavity can be used to provide visualization. The two strands of the guide 102 near the hole 106 can be brought together by a sliding knot or clip to close the hole 106. The guide 102 can be used prior to gastrotomy to help tension the stomach wall and close the hole later and/or for aiding in guiding the closure device as described above.

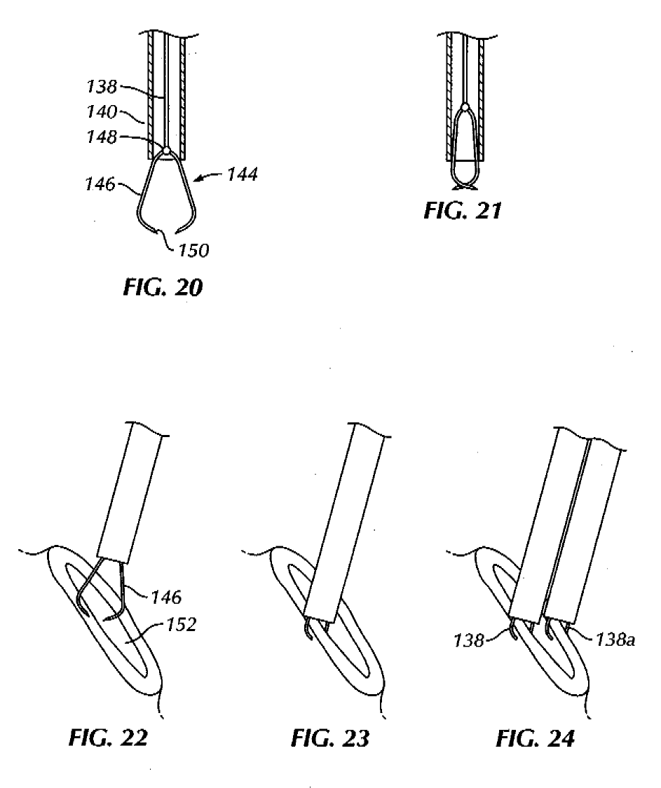

FIGS. 16-19 illustrate a pull-to-open catheter 118 that can be advanced through the mouth through a guide catheter 120 to a stomach hole to place the catheter and anchor it next to the hole. As shown, an anchor element 124 is disposed at the distal end of the catheter 118. The anchor element 124 can include opposed pinch arms 126 that are formed from a spring wire and that can be compressed when pulled proximally into the guide catheter 120 as shown in FIG. 17 to distance the distal ends 130 of the pinch arms 126 from each other. With the distal ends apart as shown in FIG. 18, the distal ends can be advanced to tissue on one side of a tissue hole 132, and then the guide catheter 120 is moved proximally relative to the pinch arms 126 (and indeed is removed from the body) to move the distal ends 130 under material bias toward each other, pinching tissue between them to thereby anchor the pull-to-open catheter 118 adjacent the hole 132 (FIG. 19). A second catheter 118a is advanced and engaged with tissue on the opposite side of the hole as shown in FIG. 19 and the catheters 118, 118a used in accordance with principles above to purse the tissue and guide a closure device to the pursed hole to close it.

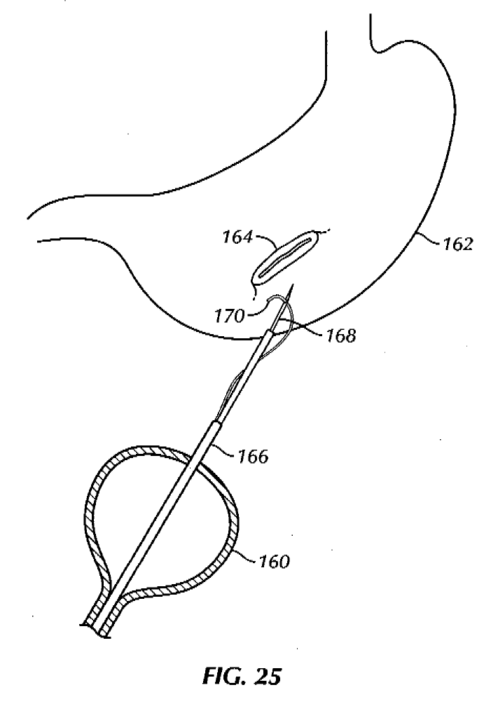

FIGS. 20-24 illustrate a pull-to-close catheter 138 that can be advanced through the mouth through a guide catheter 140 to a stomach hole to place the catheter and anchor it next to the hole. As shown, an anchor element 144 is disposed at the distal end of the catheter 138. The anchor element 144 can include opposed pinch arms 146 that are pivotably joined together at a pivot joint 148 so that when pulled proximally into the guide catheter 140 as shown in FIG. 21 the distal ends 150 of the pinch arms 146 are urged toward each other. With the distal ends apart as shown in FIG. 20, the distal ends can be advanced to tissue on one side of a tissue hole 152, and then the guide catheter 140 is moved distally relative to the pinch arms 146 to move the distal ends 150 toward each other, pinching tissue between them to thereby anchor the pull-to-open catheter 138 adjacent the hole 152 (FIG. 23). A second catheter 138a is advanced and engaged with tissue on the opposite side of the hole as shown in FIG. 24 and the catheters 138, 138a used in accordance with principles above to purse the tissue and guide a closure device to the pursed hole to close it.

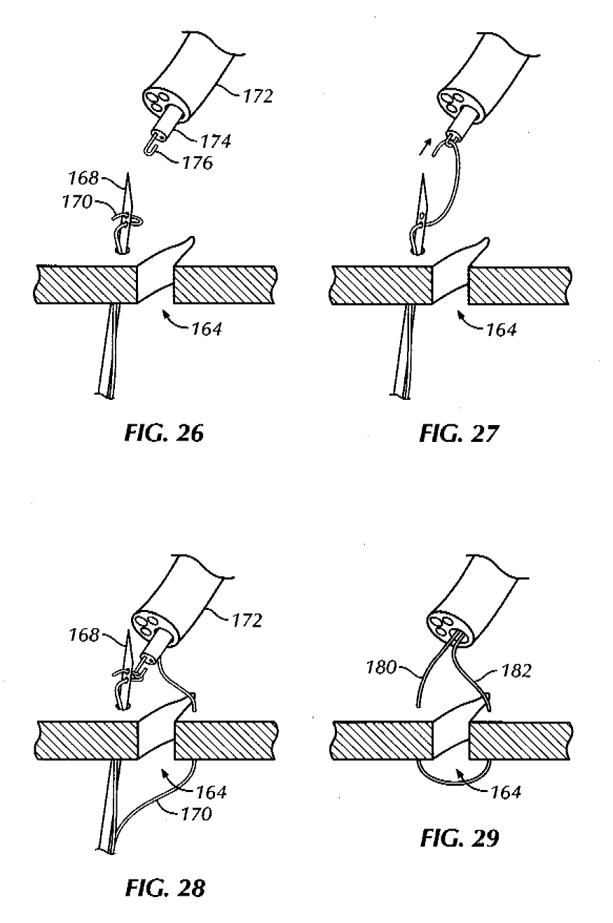

As described above, FIGS. 10-15 illustrate placement of the present guides from outside the stomach. FIGS. 25-29 provide additional details on one implementation of this feature in which guides are advanced through the bladder 160 to the stomach 162 to close a hole 164 therein.

An endoscope 166 is advanced through a natural orifice into the bladder 160. A hole is formed in the bladder and the endoscope advanced out of the bladder hole toward the hole 164 of the stomach. A suture needle 168 which is engaged with a suture thread 170 is advanced out of the endoscope 166.

FIG. 26 shows that the suture needle 168, under visualization of the endoscope 166, is pushed through the stomach adjacent the hole 164. A second endoscope 172 is advanced through the mouth into the stomach, and a suture hook catheter 174 with distal suture hook 176 or other grasping implement advanced out of the distal end of the second endoscope 172 to grasp the suture thread 170 (FIG. 27). The thread 170 is retrieved through the second endoscope 172 and the needle 168 retracted to the exterior of the stomach.

FIG. 28 illustrates that the above process is repeated on the other side of the stomach hole 164, advancing the needle 168 with suture thread 170 through the stomach wall in what might be regarded as another stitch. The thread 170 is grasped again and pulled into the second endoscope 172 to form the loop shown in FIG. 29, in which the looped thread establishes first and second guides 180, 182 for use in accordance with principles described above.

While the particular SYSTEM AND METHOD FOR TRANSLUMENAL CLOSURE IN NATURAL ORIFICE SURGERY is herein shown and described in detail, it is to be understood that the subject matter which is encompassed by the present invention is limited only by the claims.

What is claimed is:

1. A method comprising:
engaging at least one guide with stomach tissue adjacent a hole in the tissue, the guide extending at least into the esophagus;
advancing a closure device through the mouth and esophagus into the stomach over the guide;
retracting the guide to move tissue contiguous to the hole into a pursed configuration; and
actuating the closure device to hold the tissue in the pursed configuration.

2. The method of claim 1, wherein the closure device uses non-mechanical means to fuse tissue in the pursed configuration.

3. The method of claim 1, wherein the closure device uses at least one mechanical fastener to hold tissue in the pursed configuration.

4. The method of claim 3, wherein the fastener is a staple.

5. The method of claim 3, wherein the fastener is a suture.

6. The method of claim 3, wherein the fastener is a T-anchor.

7. The method of claim 1, comprising using at least two guides to move tissue contiguous to the hole into a pursed configuration.

8. The method of claim 1, wherein the guide is a catheter.

9. The method of claim 1, wherein the guide is a wire.

10. The method of claim 1, wherein the guide is a suture thread.

11. An assembly, comprising:
a closure device configured for adhering tissue together using mechanical or non-mechanical means; and
at least one guide configured for engagement with body tissue while extending out of a patient's natural orifice, the closure device being formed with a guide channel for receiving the guide therein to facilitate advancing the closure device through a natural orifice to a tissue opening to be closed.

12. The assembly of claim 11, wherein the closure device includes opposed closure surfaces between which tissue can be pulled by pulling the guide.

13. The assembly of claim 11, wherein the closure device uses non-mechanical means to hold tissue together.

14. The assembly of claim 11, wherein the closure device uses at least one staple to hold tissue together.

15. The assembly of claim 11, wherein the closure device uses at least one suture to hold tissue together.

16. The assembly of claim 11, wherein the closure device uses at least one T-anchor to hold tissue together.

17. A method for closing a hole in tissue, comprising:
advancing at least one guide to the hole;
engaging the guide with tissue adjacent the hole;
advancing a closure device through a natural orifice over the guide to the tissue adjacent the hole;
moving the guide to purse tissue together in a pursed configuration; and
actuating the closure device to adhere tissue in the pursed configuration.

18. The method of claim 17, wherein the closure device uses non-mechanical means to fuse tissue in the pursed configuration.

19. The method of claim 17, wherein the closure device uses at least one mechanical fastener to hold tissue in the pursed configuration.

20. The method of claim 19, wherein the fastener is a staple.

21. The method of claim 19, wherein the fastener is a suture.

22. The method of claim 19, wherein the fastener is a T-anchor.

23. The method of claim 17, comprising using at least two guides to move tissue contiguous to the hole into a pursed configuration.

24. The method of claim 17, wherein the hole is in the alimentary tract.

25. The method of claim 24, wherein the hole is in the stomach.

26. The method of claim 25, wherein the guide is engaged with the hole from outside the stomach and the closure device is advanced through the mouth.

27. The method of claim 25, wherein the guide is engaged with the hole from the mouth and the closure device is advanced through the mouth.

28. The method of claim 17, wherein the hole is in the vagina.

29. The method of claim 17, wherein the hole is in the uterus and the natural orifice is the vagina.

30. The method of claim 17, wherein the hole is in the stomach and the natural orifice is the urethra.

31. The method of claim 17, wherein the hole is in the urethra and the natural orifice is the urethra.
